(19) United States
(12) Patent Application Publication
(10) Pub. No.: US 2022/0249727 A1
(43) Pub. Date: Aug. 11, 2022

(54) ULTRAVIOLET LAMP

(71) Applicant: AMMI LLC., Westminster, CA (US)

(72) Inventor: Jagath Anton Swaris, Burlington (CA)

(73) Assignee: AMMI LLC., Westminster, CA (US)

(21) Appl. No.: 17/174,321

(22) Filed: Feb. 11, 2021

(51) Int. Cl. A61L 9/20; A61L 2/10; F21S 4/00

(52) U.S. Cl. CPC A61L 9/20; A61L 2202/16; F21S 4/00; A61L 2/10

(57) ABSTRACT

An ultraviolet lamp. There is a lamp housing; a plurality of rows of ultraviolet (UV) light emitters, coupled to the housing and disposed linearly and longitudinally along a single face of the lamp housing; wherein each row includes an array of clusters of UV light emitters, each cluster including a diverse set of UV light emitters, wherein the light emitters are diverse in the frequency spectrum of light emission between at least two of the emitters in the cluster; wherein the clusters of each row have consistent light emitter placement configuration with respect to clusters within the same row; and wherein the clusters of a first row have a different configuration than the clusters of a second row; and a power source coupled to the lamp housing opposite the single face and in functional communication with the UV light emitters.

### CROSS-REFERENCE TO RELATED APPLICATIONS

None.

### BACKGROUND OF THE INVENTION

### Field of the Invention

The present invention relates to ultraviolet light, specifically to ultraviolet lamps that disinfect areas with ultraviolet light.

### Description of the Related Art

Infections are known to spread through microorganisms such as bacteria, fungi, viruses, protozoa. Such microorganism can exist in room environments in the air or on exposed surfaces. When a person comes into contact with the pathogens, they are at risk of developing an infection.

In the related art, it has been known to use ultraviolet (UV) light for disinfection and sterilization. In 1892, Professor Marshall Ward demonstrated that it was primarily the UV portion of the spectrum of light that had the ability to inactivate the DNA of pathogens thereby making them unable to multiply. UV decontamination systems may sterilize the media or surface by exposing it to ultraviolet radiation of a sufficient power and for a sufficient exposure time to destroy by dimerization of thymine pairs the DNA molecular structure of bacteria, viruses, protozoa, and other organisms, which may be present in the media.

Medical uses of UV light include sterilization of surfaces and air without the use of chemicals. Ultraviolet germicidal radiation has been successfully used in purification and sterilization systems for various media, such as air, water, and food. Ultraviolet germicidal radiation has proven effective at destroying antibiotic resistant organisms. UV radiation is a particularly useful tool in the fight against hospital acquired infections superbugs such as Clostridium Difficile (C. Diff), Methicillin Resistant Staphylococcus aureus (MRSA), Vancomycin Resistant Enterococcus (VRE), etc.

Conventional decontamination devices include an ultraviolet light source that broadcasts ultraviolet light towards all exposed surfaces in a room to be decontaminated. Such an apparatus is positioned at a desired location within the room and an “on” button is pushed to turn the ultraviolet light source on. A delay circuit can be provided to the decontamination device to provide the operator sufficient time to exit the room after pushing the on button to avoid exposing the operator to the ultraviolet light emitted.

As an extra precaution, a sign can be placed in front of the door leading into the room instructing people not to enter the room while the decontamination device is active. Further, a remote control can be used by the operator to activate the decontamination device from outside of that room once the operator has exited the room. But utilizing all of these precautionary measures requires a remote control, a warning sign, etc. to be transported as separate items from location to location to decontaminate different rooms, which is inconvenient to the operator. Further, it is likely that one or more of such objects will be lost as the decontamination device is repeatedly transported and deployed.

Some improvements have been made in the field. Examples of references related to the present invention are described below in their own words, and the supporting teachings of each reference are incorporated by reference herein:

U.S. Pat. No. 10,010,633, issued to Trapani, discloses a sterilization system consisting of a mobile emitter, a sensing subsystem and a data logging subsystem is described. The emitter has one or more UV emitting lamps or devices. The sensing system comprises at least one remote UV sensor and at least one door sensor. The door sensor comprises a safety shut off door detector and may contain an emergency stop detector and arming detector to protect people from being exposed to UV energy. The system has a remote control for starting, stopping and setting system parameters which include but are not limited to: treatment time, dosage, room size, room number, unit number, floor, facility name, operator name, operator identification number, password, default dosage values, dosage, and patient identification number. The number of treatments per unit of time can be maximized because of the use of incident light measurement.

U.S. Pat. No. 10,092,665, issued to Lyslo et al., discloses UV hard-surface disinfection system that is able to disinfect the hard surfaces in a room, while minimizing missed areas due to shadows by providing multiple UV light towers that can be placed in several areas of a room such that shadowed areas are eliminated and that can be transported by a cart that is low to the ground such that the towers may be loaded and unloaded easily by a single operator. The system is able to be controlled remotely, such that during activation of the system, no operator is present, and to automatically cut power to all towers in the event that a person enters the room.

U.S. Patent Application Publication No.: 2018/0185533, by Lalicki et al., discloses control systems for disinfecting light systems and methods of regulating operations of disinfecting light systems are disclosed. The control system may include a controller operably coupled to a disinfecting light fixture illuminating a space. The controller may regulate an operation of the disinfecting light fixture by adjusting an amount of disinfecting energy provided to the space by the disinfecting light fixture and/or adjusting an amount of illuminating light provided to the space by the disinfecting light fixture. The amount of disinfecting energy may be adjusted based on data relating to the space including disinfecting energy provided to the space, a bacterial load of the space, and/or environ mental characteristic(s) of the space. Additionally, the amount of illuminating light may be adjusted based on data relating to the space including environmental characteristic(s) of the space, and/or a predetermined operational schedule for the space.

International Application Published Under the Patent Cooperation Treaty (PCT) No: WO 2016/210399, by Dayton, discloses provided is a decontamination apparatus and method involving a base, and a plurality of sources that each emit UVC light to render a target object pathogen reduced. A plurality of adjustable supports couple the sources to the base, and a controller is coupled to the base to be operatively connected to the sources to control emission of the UVC light. A housing is removably installed on the base to protect the plurality of sources. A remote control is provided to the housing and includes a user interface that receives a input from a user and transmits a control instruction to the controller based on the input received, resulting in desired operation of the sources by the controller.

Canadian Patent No: 2931403, issued to Stibich et al., discloses systems are disclosed which include processor-executable program instructions for receiving data regarding characteristics of a room in which one or more disinfection sources are arranged and determining, based on the received data, individual operating parameter/s for the one or more disinfection sources. Other systems are provided which include processor-executable program instructions for discerning, for each of a plurality of disinfection sources, a target location, region, object or surface within a room in which the disinfection sources are arranged. The systems further include program instructions for comparing the target locations/regions/objects/surfaces and executing corrective action/s upon detecting two or more locations/objects/surfaces are within a predetermined distance of each other and/or upon detecting two or more regions overlap.

European Patent Application Publication No: 3668165, by Ufkes, discloses a portable UV-C disinfection apparatus, method, and system for ultraviolet germicidal irradiation. UV-C emitters may be coupled to an array housing having a planar array surface in a vertical configuration. UV-C sensors are configured to measure the amount of UV-C light or near UV-C light from a target surface. A controller may be configured to engage with the UV-C sensors to determine the amount of UV-C radiation collected by the UV-C sensors. The controller includes instructions stored on a memory according to the amount of UV-C radiation collected corresponding to an effective kill-dose for surface disinfection. The improved apparatus, method, and system reduces exposure time by varying the intensity and wavelength of the UV-C administered, while concurrently reducing UV overexposure to surfaces by administering radiation through a rotational zonal application.

Japanese Patent No: 2014523257, discloses a sterilization system with a moving radiator, a detection subsystem and a data logging system is proposed. One or a plurality of devices such as ultraviolet lamps are used as the radiator. As the detection subsystem, one or more ultraviolet sensors and one or more door sensors are used. As the door sensor, a safety stop door state detector is used, and an emergency stop instruction detector and an operable state detector are provided to suppress exposure of the human body to ultraviolet radiation. In this system, start, stop, and system parameter setting are performed with a remote controller. System parameters are treatment time, dose, room size, room number, facility number, floor, facility name, operator name, operator identification number, password, dose default value, presence/absence of irradiation, patient identification number, etc. Since incident ultraviolet ray measurement is used, the number of treatments per unit time can be increased.

The inventions heretofore known suffer from a number of disadvantages which include: producing more heat at higher UV outputs; not lasting as long; being less reliable; covering a smaller area; not being a modular device allowing for daisy-chaining and/or parallel networks of devices to cover a larger area; not being able to regulate the strength of the radiation without physical access to the device; not being configurable to target specific microorganisms; not radiating all three types of UV radiation in a single device; not replacing multiple sanitizing solutions with a single device; being less effective at sanitizing; and having less even distribution of radiation.

What is needed is an ultraviolet lamp that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available ultraviolet lamps for sterilization. Accordingly, the present invention has been developed to provide an ultraviolet lamp.

In one embodiment of the invention, there may be an ultraviolet lamp. The ultraviolet lamp may comprise: a lamp housing; a plurality of rows of ultraviolet (UV) light emitters, that may be coupled to the housing and/or may be disposed linearly and/or longitudinally along a single face of the lamp housing; wherein each row may include an array of clusters of UV light emitters, each cluster may include a diverse set of UV light emitters, wherein the light emitters may be diverse in the frequency spectrum of light emission between at least two of the emitters in the cluster; wherein the clusters of each row may have consistent light emitter placement configuration with respect to clusters within the same row; and/or wherein the clusters of a first row may have a different configuration than the clusters of a second row; and/or a power source that may be coupled to the lamp housing opposite the single face and/or in functional communication with the UV light emitters.

In another embodiment of the invention, the UV emitters may be selected from the group consisting of: UVA LED lights; UVB LED lights; and/or UVC LED lights. Further, in an embodiment of the invention, the ultraviolet lamp may further comprise a third row, wherein: the clusters of the first row may include two UVC LED lights, one UVA LED light, and/or one UVB LED light; the clusters of the second row may include two UVA LED lights, one UVB LED light, and/or one UVC LED light; and/or the clusters of the third row may include two UVB LED lights, one UVA LED light, and/or one UVC LED light. Also, in one embodiment of the invention, the ultraviolet lamp may further comprise a wireless controller, that may be in wireless communication with the UV emitters, and/or in communication with the power source, wherein the wireless controller may have an on-mode and/or an off-mode.

Additionally, in one embodiment of the invention, the UV emitters may be selectably dimmable at least one of: individually, by cluster, or by row. In yet another embodiment of the invention the ultraviolet lamp may further comprise: an occupancy sensor, that may be in communication with the power source, wherein in a negative mode no occupants may be sensed by the occupancy sensor, and/or in a positive mode occupants may be sensed by the occupancy sensor, and/or a control module that may include instructions for automatically dimming the UV emitters when the occupancy sensor may be in a positive mode but not necessarily dimming all emitters the same amount. Furthermore, in one embodiment of the invention, the ultraviolet lamp may further comprise a printed circuit board that may be disposed within the housing, that may be in communication with the power source, and/or that may be coupled to the UV emitters. More, in one embodiment of the invention, the ultraviolet lamp may further comprise a plurality of heat sinks that may be disposed behind the printed circuit board opposite the UV emitters. Too, in one embodiment of the invention, the ultraviolet lamp may further comprise a plurality of optics that may be disposed in front of the UV emitters, opposite the heat sinks.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawing(s). It is noted that the drawings of the invention are not to scale. The drawings are mere schematics representations, not intended to portray specific parameters of the invention. Understanding that these drawing(s) depict only typical embodiments of the invention and are not, therefore, to be considered to be limiting its scope, the invention will be described and explained with additional specificity and detail through the use of the accompanying drawing(s), in which:

FIG. 1 is a front perspective view of an ultraviolet lamp, according to one embodiment of the invention;

FIG. 2 is a front perspective exploded view of an ultraviolet lamp, according to one embodiment of the invention;

FIG. 3 is a top plan view of a printed circuit board showing clusters of ultraviolet light emitters, according to one embodiment of the invention;

FIG. 4 is a front perspective view of an ultraviolet light emitter, according to one embodiment of the invention;

FIG. 5 is a chart of the electromagnetic spectrum, according to one embodiment of the invention;

FIG. 6 is a graph of ultraviolet light emission by frequency showing percentages of spectral irradiance based on wavelength in nanometers, according to one embodiment of the invention;

FIG. 7 illustrates a schematic diagram of electronic components of an ultraviolet lamp, according to one embodiment of the invention; and

FIG. 8 illustrates a schematic diagram of electronic components of an ultraviolet lamp, according to one embodiment of the invention.

### DETAILED DESCRIPTION OF THE INVENTION

For the purposes of promoting an understanding of the principles of the invention, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to an “embodiment,” an “example” or similar language means that a particular feature, structure, characteristic, or combinations thereof described in connection with the embodiment is included in at least one embodiment of the present invention. Thus, appearances of the phrases an “embodiment,” an “example,” and similar language throughout this specification may, but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc.

Each statement of an embodiment, or example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps. “Comprising” is to be interpreted as including the more restrictive terms “consisting of” and “consisting essentially of.”

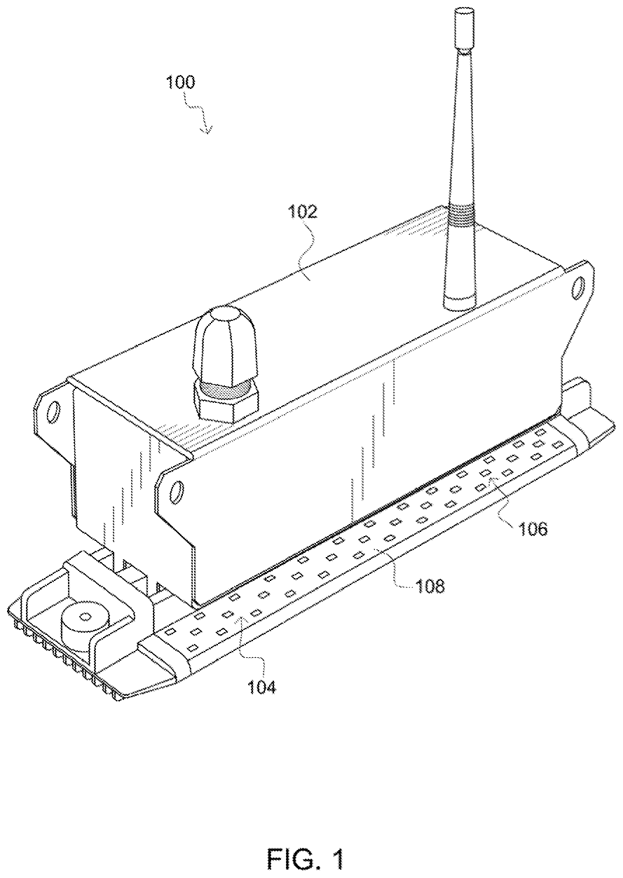

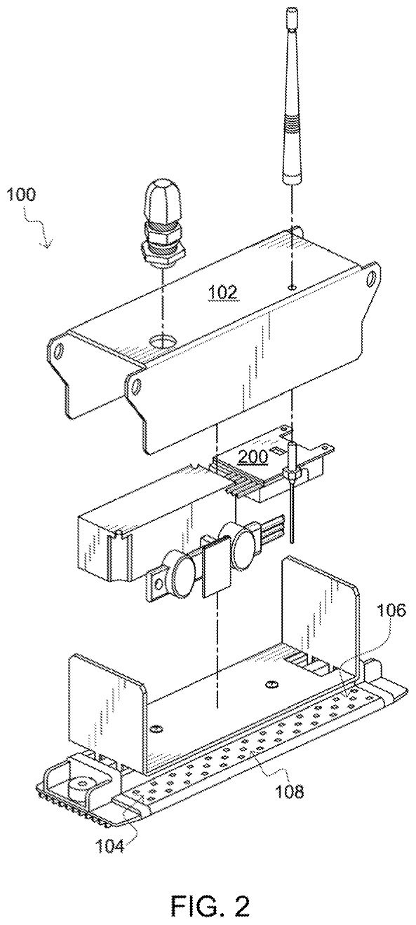

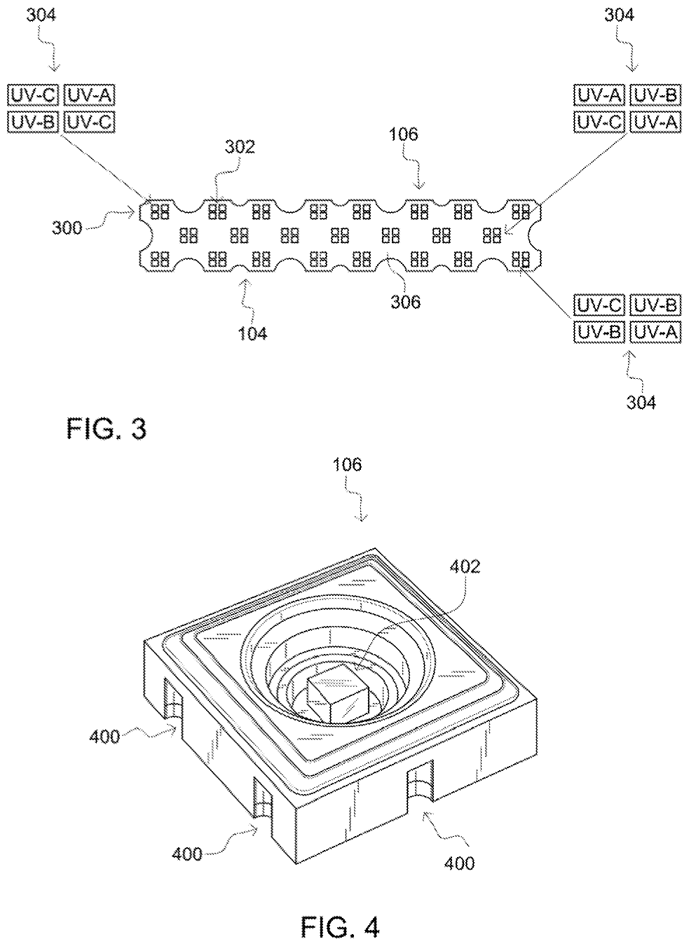

FIG. 1 is a front perspective view of an ultraviolet lamp, according to one embodiment of the invention. There is shown an ultraviolet lamp 100. The ultraviolet lamp 100 includes a lamp housing 102. Disposed along a single face 108 of the lamp housing 102 is a plurality of rows 104 of ultraviolet light emitters 106 (the backside of the circuit board holding the emitters is illustrated in FIGS. 1 and 2, while the emitters themselves are illustrated in FIG. 3).

The illustrated lamp housing 102 houses the components of the ultraviolet lamp 100. The lamp housing 102 is configured to secure, contain, and/or protect the components of the lamp 100. The illustrated housing 102 is substantially rectangular, however, the housing may have any size and/or shape for securing, containing, and/or protecting the lamp 102. The housing 102 also allows for mounting the device 100. In addition, the housing 102 is substantially durable. As a result, the housing 102 may be comprised of materials such as, but not limited to: metal, plastic, and other synthetics.

The illustrated housing 102 includes the single face 108. Disposed linearly and longitudinally along a front of the single face 108 is a plurality of ultraviolet light emitters 106, which may be dimmable UV light emitters, such as but not limited to dimmable UV LEDs. The ultraviolet light emitters 106 may be UVA LED lights, UVB LED lights, and/or UVC LED lights. For instance, the ultraviolet light emitters may be such as a UV Ultraviolet LED 5 mm 400 nm 3.5V, by xUmp.com, 3710 Industry Ave., Suite 206, Lakewood, Calif. 90712, which is incorporated by reference herein. As shown, the light emitters 106 are disposed in rows 104. The light emitters 106 are shown coupled to the housing 102. The light emitters 106 are coupled to the housing 102 so that they irradiate away from the housing 102.

In operation of one embodiment, there is a targeted sterilizing device, such as an ultraviolet lamp 100, that targets certain germs/viruses. The device includes a plurality of UVA, UVB, and UVC light emitters that are electronically controlled independently so that the different types of emitters may emit at different output strengths. The device can target anything that UV can target, anything from UVA, B. and C. Anything vulnerable to those wavelengths can be sterilized. It can sterilize an environment, objects, and in some cases humans as well. One can regulate the strength of the radiation. For example, if there are no humans present, one can cause the device to radiate at a higher strength. If there are humans present, one can utilize the controller to limit emission to a certain strength at particular wavelengths that is suitable for human presence. One can pick the radiation types and amounts that you want to radiate. One can target the space/area strategically with a single device to match up the deployed radiation levels to match the intended space. One can modify the distribution and the strength of the radiation to match the desired levels of radiation at a particular surface(s) at a particular distance. One can automatically change the radiation on detection of humans being present, but instead of just shutting off the radiation or dropping the level of radiation at all emitted wavelengths, one can tailor the reduction differently by reducing the emission levels disparately between the multiple types of emitters.

In one nonlimiting embodiment, the device includes optics, electronics, heat sinking, and a housing. The optics include the emitters and how the emitters are arranged on the front surface of the device (e.g. on a PCB board). You have optics to distribute the light to the target area. The emitters (e.g. LEDs) are of three different types, UVA, UVB, and UVC and are located on the front of the device (e.g. PCB board). The arrangement of the emitters is designed to target a maximum area of exposure. Wherein the emitters are LEDs, there is a driver to drive the LEDs, which driver is basically a power supply. The power supply generally includes an AC to DC converter, the DC power goes into the board and converts the electrical energy into light radiation.

When the current passes through the LED, light and heat are generated. If the device fails to extract heat from the emitters the LEDs will degrade. The device will generally utilize three ways of removing the heat (conduction, convection, and radiation). Heat sinks are used to conduct and convect away the heat. The optics (e.g. a transparent faceplate(s) of the device that covers the emitters and protects them from being impacted/scratched) are selected to not reflect IR radiation back into the LEDs (e.g. fused silica, crystalline quartz, N-BK7 (a borosilicate crown glass), sapphire, zinc selenide, and zinc sulfide) and also to not reflect the UV emissions from the emitters (e.g. UV fused silica, N-BK7, magnesium fluoride, calcium fluoride, sodium chloride, potassium bromide, and sapphire). The illustrated housing protects the components, provides space for the heat sinks, and allows for mounting the device. The heat sinks are generally disposed behind the PCB and draw heat out of the LEDs.

In one non-limiting embodiment, there is a wireless transponder that can be accessed through a phone application that allows a user to control the device remotely (e.g. issue commands to the controller to activate/deactivate and/or attenuate the emitters, such as but not limited to by type of emitter). Motion sensors and other sensors may be in communication with the application and thereby used to further control/manage the device. The LEDs are independently controllable and thereby the device is able to be modulated with respect to how much of each type of radiation is output. The LEDs are generally spaced so that a uniform distribution of radiation reaches a target region.

This uniform distribution of radiation combined with the ability to individually attenuate/modulate the emission of the various emitters and/or types of emitters is particularly advantageous as there are many applications of sanitizing situations wherein specific doses of radiation must be administered for specific time periods to specifically sized surfaces in particularly sized rooms and variations/fluxuations in the actual dosing and/or timing may be detrimental and/or dangerous. Accordingly, one may, with a single device, serve a multiplicity of uses across a wide spectrum of markets and industries (e.g. medical device manufacturing, food processing, room sanitization, microbe growth control, classroom settings, events, shower/changing rooms, pet stores/services, offices, etc.). This allows for enhanced scalability and reduced manufacturing costs, as customization to the particular need is done via programming and selecting the number of units to purchase, instead of requiring custom hardware components.

FIG. 2 is a front perspective exploded view of an ultraviolet lamp, according to one embodiment of the invention. There is illustrated an ultraviolet lamp 100 with a lamp housing 102. Disposed along a single face 108 of the lamp housing 102 is a plurality of rows 104 of ultraviolet light emitters 106. A power source with heat sinks 200 is disposed within the lamp housing 102.

As shown, the lamp housing 102 is disposed over the power source 200. The lamp housing 102 protects and stores the power source 200. The power source 200 is coupled to the lamp housing 102 opposite the single face 108 and is in functional communication with the UV light emitters 106. The power source 200 provides power to the light emitters 106. Accordingly, the power source 200 may be an electronic power source such as, but not limited to, a battery. The power source 200 may be an AC to DC converter. For example, the power source 200 may include a battery such as a lithium battery, a lithium ion battery, or an alkaline battery. However, the power source 200 may be any device for providing power to the ultraviolet light emitters 106. The power source 200 may be a driver to drive the UV light emitters 106. The power source 200 may be a driver such as an LED7707 LED Driver, by Digi-Key Electronics, 701 Brooks Avenue South, Thief Rive Falls, Minn. 56701, and/or the HitLights 40 Watt Dimmable LED Driver with 12 V Magnetic Power Supply by HitLights at www.hitlights.com (accessed on Dec. 23, 2020), which is incorporated by reference herein.

The controller may include one or more wireless smart dimmer switches and/or one or more multi-channel dimmers, such as the components found within the Philips Hue Wireless Dimmer Switch with Remote by Philips North America Corporation of 3000 Minuteman Road M/S 109. Andover, Mass. 01810. Accordingly, the device may be remotely controlled and via such remote control may be remotely configured to produce varying spectral emission profiles to create custom emission spectrum for particular uses without any changes to hardware (e.g. without changing out any emitters or replacing the device with another different device).

FIG. 3 is a top plan view of a printed circuit board showing clusters of ultraviolet light emitters, according to one embodiment of the invention. There is shown a plurality of rows 104 of ultraviolet light emitters 106. The plurality of rows 104 of emitters 106 are disposed on a printed circuit board (PCB) 306 in an array 300. The ultraviolet light emitters 106 are also shown disposed as clusters 302 of emitters 106, where each cluster 302 is a diverse set 304 of emitters 106.

The illustrated ultraviolet light emitters 106 are disposed in three rows 104. Each row 104 includes an array 300 of clusters 302 of UV lights 106. As shown, each cluster 302 includes a diverse set 304 of UV lights 106. The clusters 302 are diverse sets 304 in that each set 304 includes a different frequency spectrum of light emission between at least two of the emitters 106 in the cluster 302.

Further, the illustrated clusters 302 of light emitters 106 have consistent light emitter 106 placement in each row 104 with respect to clusters 302 of the same row. In one embodiment, the clusters 302 of a first row 104 have a different configuration that the clusters 302 of a second row 104. For instance, the clusters 302 of a first row 104 may include two UVC LED light emitters 106, one UVA LED light emitter 106, and one UVB LED light (e.g. CCAB); the clusters 302 of a second row 104 may include two UVA LED light emitters 106, one UVB LED light emitter 106, and one UVC LED light emitter 106 (e.g. AABC); and the clusters 302 of a third row 104 may include two UVB LED light emitters 106, one UVA LED light emitter 106, and one UVC LED light emitter 106 (e.g. BBAC). The ultraviolet light emitters 106 may have any arrangement designed to target a maximum area of exposure. In one embodiment, there is a UV LED light emitter 106 combination of A, B and C that are independently controllable and positioned and spaced to provide an even distribution of radiation to a target zone.

While the illustrated clusters and rows include four emitters for each cluster and three rows of clusters, it is understood that various embodiments may include clusters and rows of other sizes, such as but not limited to clusters of 2, 3, 5, 6, 7, and/or 8+ emitters/rows on a single device as well as diversity of cluster sizes between rows. As a non-limiting prophetic example, there may be a device wherein Rows 1 and 3 have 4-emitter clusters (AABC and BBAC, respectively), while Rows 2 and 4 have 3-emitter clusters (CCA and CCB, respectively), and Row 5 has 5-emitter clusters (AABCC). The clustering of diverse types of emitters allows for the device to produce complex spectral output that is uniform across a region since the clusters effectively operate as point sources of the output for most sanitizing purposes.

In one non-limiting embodiment, there are a plurality of rows and at least two of the rows include a different configuration of clusters. It may be that the configuration of rows is mirrored (e.g. using configuration numbers in row sequence, the following would be mirrored row configurations: 1, 2, 1; 1, 2, 3, 3, 2, 1; or 1, 2, 3, 2, 1). It may be that the configuration of rows is repeated (e.g. 1, 2, 1, 2; 1, 2, 3, 1, 2, 3). It may be that the clusters are ordered in a 2×2 grid (i.e. top-left, top-right, bottom-left, bottom-right) and it may be that a grid of A, A, B, C is functionally equivalent to a grid of C, A, A, B; to a grid of B, C, A, A; and to a grid of A, C, A, B; and etc., as the cluster itself is functioning as a point source of emission. It may be that the point-source proportion and row-wise distribution of the diverse emitters is more important than the exact location within each cluster of the diverse emitters.

The clusters and rows also allow for spacing between the groups of emitters for more effective heat removal (e.g. via functionally coupled heat sink). This allows for powerful and efficient emitters to be utilized while maintaining even spectral emission distribution over a region, thereby resulting in customizable and predictable sanitization outcomes.

Also shown, the light emitters 106 are coupled to a printed circuit board (PCB) 306. The PCB 306 may be disposed within the housing (See e.g., FIG. 1, Item 102) and in communication with the power source (See e.g., FIG. 2, Item 200). Accordingly, the printed circuit board 306 may be an electronic circuit consisting of thin strips of a conducting material, such as copper, which have been etched to a flat insulating sheet, and to which integrated circuits and other components are attached. For example, the PCB 306 may be such as a PCB Assembly of Sierra Circuits, 1108 West Evelyn Avenue, Sunnyvale, Calif. 94086, which is incorporated by reference herein. Accordingly the power source (Se e.g., FIG. 2, Item 200) may provide DC power to the PCB 306 to convert electrical energy into light radiation. Furthermore, the light emitters 106 may be spaced along the PCB 306. The light emitters 106 may be spaced along the PCB so that a uniform distribution of radiation reaches a target region.

FIG. 4 is a front perspective view of an ultraviolet light emitter, according to one embodiment of the invention. There is illustrated an ultraviolet light emitter 106. The ultraviolet light emitter 106 has a plurality of heat sinks 400 disposed behind the ultraviolet light emitter 106. The ultraviolet light emitter 106 also has an optic 402 disposed in front of the ultraviolet light emitter 106 opposite the heat sinks 400.

The illustrated ultraviolet light emitter 106 may be a UVA LED light, a UVB LED light, or a UVC LED light. The ultraviolet light emitter 106 may be an excimer lamp. For instance, the ultraviolet light emitter may be an ultraviolet light such as a UVC UV Germicidal Lamp UV Sterilizer Disinfection Light Bulb E27 72LED, by Tuscom, 615 Queen City Avenue, Tuscaloosa, Ala. 35401, which is incorporated by reference herein. Also, in one embodiment of the invention, the light emitter 106 may be dimmable. For example, the light emitter 106 may be a dimmable light such as LED 13695 Satco LED Dimmable A19 Glass E26 Base, by Satco, 110 Heartland Blvd., Brentwood, N.Y. 11717, which is incorporated by reference herein.

As shown, the plurality of heat sinks 400 are disposed behind the ultraviolet light emitter 106. Accordingly, the heat sinks 400 may be disposed behind the printed circuit board (See e.g., FIG. 3, Item 306) opposite the UV emitter 106. The heat sinks 400 are shown as a plurality of grooves or channels beneath the light emitter 106. The heat sinks 400 function to absorb heat from the UV light emitter 106 and prevent the UV light emitter 106 from overheating. The heat sinks 400 conduct and convect heat away from the UV emitter 106. As a result, the heat sinks 400 may extend a life of an ultraviolet light emitter 106. As a result, the light emitter 106 and heat sinks 400 may be such as an Ohmite SV-LED 314E Heat Sink, by Ohmite Manufacturing, 27501 Bella Vista Parkway, Warrenville, Ill. 6055, which is incorporated by reference herein.

The illustrated optic 402 is disposed in front of the ultraviolet light emitter 106. Accordingly, the optic 402 is disposed in front of the UV emitter 106 opposite the heat sinks 400. The optic 402 helps to control light emitted from the light emitter 106. The optic 402 distributes light from the light emitter 106 to a target area. The optic 402 may be a piece of glass or other transparent material cut with precise angles and plane faces. The optic 402 may be any material that does not reflect infrared radiation back into the light emitter 106.

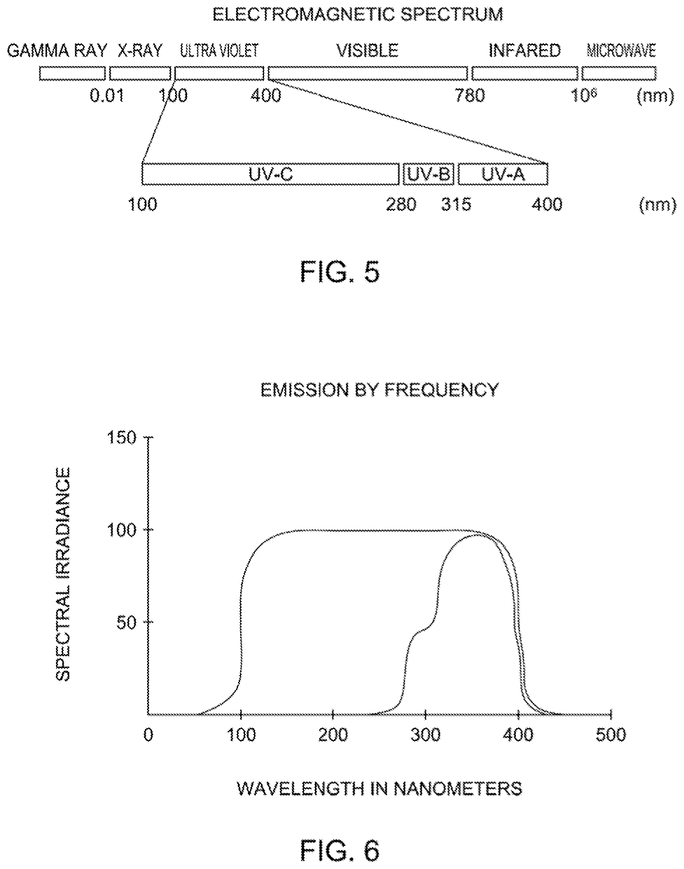

FIG. 5 is a chart of the electromagnetic spectrum, according to one embodiment of the invention. There is shown a range of the electromagnetic spectrum. The illustrated electromagnetic spectrum shows a range of ultraviolet light with wavelengths from 100 nanometers (nm) to 400 nanometers. Ultraviolet light is produced by the sun and by special lamps. Ultraviolet light is broken down into three types of ultraviolet light: UVA; UVB; and UVC. The range of UVC light spans from 100 nm to 280 nm. UBB light has a wavelength that ranges from 280 nm to 315 nm, and UVC light has a wavelength that ranges from 315 nm to 400 nm. Accordingly, UVC light has the most energy of the three types.

FIG. 6 is a graph of ultraviolet light emission by frequency showing percentages of spectral irradiance based on wavelength in nanometers, according to one embodiment of the invention. As shown, the UV emitters are selectably dimmable at least one of: individually, by cluster, or by row.

There is illustrated a Y-axis labeled “Spectral Irradiance,” and an X-axis labeled “Wavelength in Nanometers.” A first line shows a percentage of spectral irradiance with UVC, UVB, and UVA emitters having similar emission output capabilities each going on full blast (i.e. not dimmed). A second line shows a percentage of spectral irradiance with UVC emitters almost completely shut off, UVB emitters down by about half, and UVA emitters on full blast (i.e. not dimmed). In the illustrated example, the first line may be a full-spectrum sanitizing mode wherein the emitters are not dimmed and working to maximum capacity, while the second line may be a different mode, such as but not limited to a low power consumption mode or a mode wherein people are present which may be triggered by motion sensors and/or some other triggering device/component (e.g. heat sensor, power usage sensor, programmable timer) that causes a switch from the full-spectrum mode to the second mode and/or back to the full-spectrum mode.

While a particular spectrum is illustrated, it is understood that the invention(s) described herein are capable of a plethora of modes and a plethora of output spectrums which may be customized to particular applications (e.g. targeting specific groups/types of microbes/germs/viruses). Accordingly, a single device may be utilized in a near infinite number of ways and serve a tremendous variety of purposes. This advantageously allows for a single manufacturing line to service essentially every type of UV sanitization need, including needs that change over time or that are periodic.

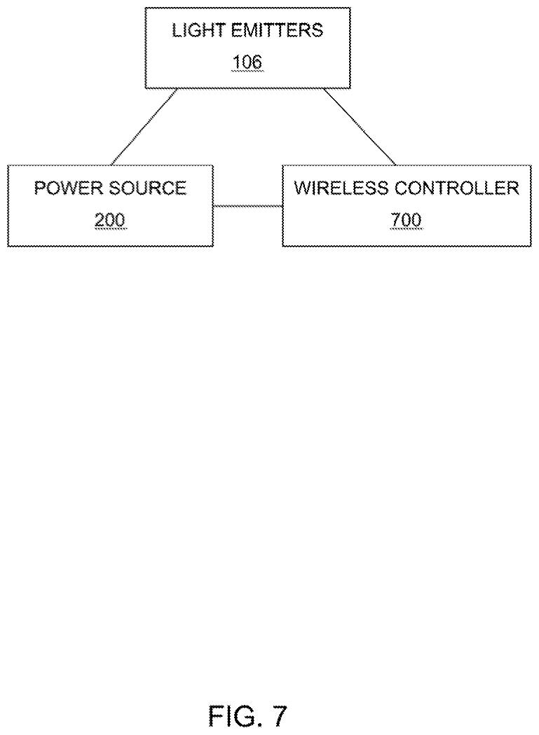

FIG. 7 illustrates a schematic diagram of electronic components of an ultraviolet lamp, according to one embodiment of the invention. There is shown a plurality of ultraviolet light emitters 106. The emitters 106 are in communication with a power source 200 and a wireless controller 700. The power source 200 is in communication with the emitters 106 and the wireless controller 700. The wireless controller 700 is in communication with the emitters 106 and the power source 200.

The illustrated light emitters 106 are in communication with a power source 200 so that the light emitters 106 are turned on when provided with power from the power source 200, and turned off when not provided with power from the power source 200. In one embodiment of the invention, the power source 200 may be a battery, such as, but not limited to a lithium, lithium ion, or alkaline battery.

As shown, the wireless controller 700 is in communication with the power source 200 and with the light emitters 106. The wireless controller 700 may have an on-mode and an off-mode to control an amount of power supplied from the power source 200 to the light emitters 106. For instance, the wireless controller 700 may function to turn the light emitters 106 on and off. Similarly, the wireless controller 700 may function to control different amounts of irradiation from the light emitters 106 so that the light emitters may be selectably dimmable. Accordingly, the wireless controller 700 may be a wireless control or wall mount switch controls by utilizing an AC/DC converter like a remote control for LED lights such as an LED Bulb UV-C Light with Remote Control, by New Sunshine, 15344a Valley Blvd., City of Industry, Calif. 91746, which is incorporated by reference herein.

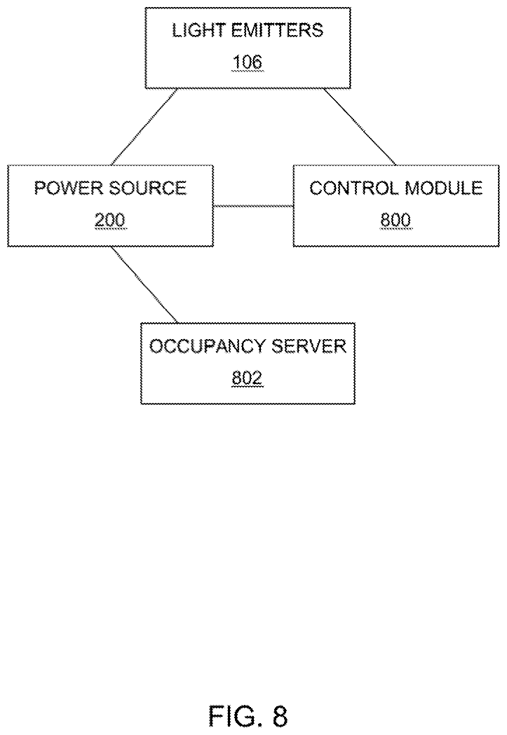

FIG. 8 illustrates a schematic diagram of electronic components of an ultraviolet lamp, according to one embodiment of the invention. There is illustrated a plurality of ultraviolet light emitters 106 in communication with a power source 200 and a control module 800. The power source 200 is in communication with the ultraviolet light emitters 106, the control module 800, and an occupancy sensor 802. The control module 800 is in communication with the light emitters 106 and the power source 200. The occupancy sensor 802 is in communication with the power source 200.

As shown, the occupancy sensor 802 is shown in communication with the power source 200. In one embodiment, if occupants are sensed by the occupancy sensor 802, then the occupancy sensor 802 is in a positive mode, and the power source 200 will not supply power to the light emitters 106. In comparison, in one embodiment, if no occupants are sensed by the occupancy sensor 802, then the occupancy sensor 802 is in a negative mode, and the power source 200 may supply power to the light emitters 106. For example, the occupancy sensor 802 may be a motion sensor such as a Homelife LED Bar Motion Sensor Light, by Homelife LED, 1771 Robson Street, Vancouver, BC V6G 1C9, Canada, which is incorporated by reference herein.

The illustrated control module 800 is shown in communication with the power source 200 and the light emitters 106. The control module 800 includes instructions for automatically dimming the light emitters 106 when the occupancy sensor 802 is in a positive mode, but not dimming all emitters 106 the same amount. For instance, the control module may selectably dim the emitters 106 individually, by cluster, or by row. For instance, the control module 800 may be an intelligent addressable lighting technology such as Bluetooth Mesh Network, or a digital addressable lighting interface such as the Integrated Sensor Control System by Eaton, 1000 Eaton Blvd., Beachwood, Ohio 44122, which is incorporated by reference herein. Accordingly, the ultraviolet lamp (Se e.g., FIG. 1, Item 100) can disinfect a space when there are no humans present. Moreover, in one embodiment, the light emitters 106 are independently controllable by the control module 800. As a result, the ultraviolet lamp (Se e.g., FIG. 1, Item 100) is able to be modulated with respect to how much of each type of radiation is output by each light emitter 106.

Further, in one embodiment, the control module 800 may include a phone app with a predetermined scheduling program instructions so that duration of disinfection can be programmed. In addition, the control module 800 may include instructions for adjusting electromagnetic radiation from 400 mw to 960 mw of the power source 200 by way of a user interface. In one embodiment, the control module 800 may include a phone app that communicates with a wireless transponder of the ultraviolet lamp (See e.g., FIG. 1, Item 100) that allows a user to control the ultraviolet lamp (See e.g., FIG. 1, Item 100). For example, the control module 800 may be a management application such as the LG ThinQ App, by LG Electronics, 201 James Record Rd. SW, Huntsville, Ala. 35824, which is incorporated by reference herein. Similarly, the control module 800 may include a phone app with a timer, such as the Windows Alarm & Clock App, by Microsoft, 15010 NE 36^{th} St., Redmond, Wash. 98052, which is incorporated by reference herein.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

For example, the UV LEDs may have variations on exact wavelength and strength. Additionally, although the figures illustrate an array of UVA, UVB and UVC light emitters, the device may have any arrangement, array, pattern, etc. of emitters. Likewise, the UV LEDs may have any shape, size and/or orientation.

It is expected that there could be numerous variations of the design of this invention. An example is that the invention may include any number of sensors, LEDs, power sources, controllers, etc. in any orientation. It is also envisioned that the housing may have any shape and/or size. Similarly, the heat sinks may have any shape and/or size. Further, the power supply may be any efficient driver and the wireless controller may be any wireless device, such as a management application of a phone app.

Finally, it is envisioned that the components of the device may be constructed of a variety of materials, such as but not limited to: plastic, glass, metal, wood, rubber, acrylic and so on. For instance, the PCB may have a metal core, FR4, and others. As another example, the LEDs may be constructed by various manufacturers, such as but not limited to: Nichia, Samsung and Seoul Semiconductor. Also, the optics may be comprised of glass, plastic and/or acrylic.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims. Further, it is contemplated that an embodiment may be limited to consist of or to consist essentially of one or more of the features, functions, structures, methods described herein.

What is claimed is:

1. An ultraviolet lamp, comprising:
a. a lamp housing;
b. a plurality of rows of ultraviolet (UV) light emitters, coupled to the housing and disposed linearly and longitudinally along a single face of the lamp housing;
i. wherein each row includes an array of clusters of UV light emitters, each cluster including a diverse set of UV light emitters, wherein the light emitters are diverse in the frequency spectrum of light emission between at least two of the emitters in the cluster;
ii. wherein the clusters of each row have consistent light emitter placement configuration with respect to clusters within the same row; and
iii. wherein the clusters of a first row have a different configuration than the clusters of a second row; and
c. a power source coupled to the lamp housing opposite the single face and in functional communication with the UV light emitters.

2. The ultraviolet lamp of claim 1, wherein the UV emitters are selected from the group consisting of: UVA LED lights; UVB LED lights; and UVC LED lights.

3. The ultraviolet lamp of claim 2, further comprising a third row, wherein:
a. the clusters of the first row include two UVC LED lights, one UVA LED light, and one UVB LED light;
b. the clusters of the second row include two UVA LED lights, one UVB LED light, and one UVC LED light; and
c. the clusters of the third row include two UVB LED lights, one UVA LED light, and one UVC LED light.

4. The ultraviolet lamp of claim 1, further comprising a wireless controller, in wireless communication with the UV emitters, and in communication with the power source, wherein the wireless controller has an on-mode and an off-mode.

5. The ultraviolet lamp of claim 1, wherein the UV emitters are selectably dimmable at least one of: individually, by cluster, or by row.

6. The ultraviolet lamp of claim 5, further comprising:
a. an occupancy sensor, in communication with the power source, wherein in a negative mode no occupants are sensed by the occupancy sensor, and in a positive mode occupants are sensed by the occupancy sensor, and
b. a control module including instructions for automatically dimming the UV emitters when the occupancy sensor is in a positive mode but not dimming all emitters the same amount.

7. The ultraviolet lamp of claim 1, further comprising a printed circuit board disposed within the housing, in communication with the power source, and coupled to the UV emitters.

8. The ultraviolet lamp of claim 7, further comprising a plurality of heat sinks disposed behind the printed circuit board opposite the UV emitters.

9. The ultraviolet lamp of claim 8, further comprising a plurality of optics disposed in front of the UV emitters, opposite the heat sinks.

10. A dimmable ultraviolet lamp, comprising:
a. a lamp housing having a rectangular front surface;
b. a plurality of dimmable UVA LED lights, disposed along the front surface of the lamp housing;
c. a plurality of dimmable UVB LED lights, disposed along the front surface of the lamp housing;
d. a plurality of dimmable UVC LED lights, disposed along the front surface of the lamp housing;
e. a control module functionally coupled to the plurality of dimmable UVA, UVB, and UVC lights and configured to control the dimming thereof; and
f. a power source, coupled to a back region of the lamp housing and in functional communication with the LED lights.

11. The dimmable ultraviolet lamp of claim 10, wherein the UV LED lights are disposed in a plurality of rows coupled to the housing and disposed linearly and longitudinally along a single face of the lamp housing;
a. wherein each row includes an array of clusters of UV lights, each cluster including a diverse set of UV lights, wherein the lights are diverse in the frequency spectrum of light emission between at least two of the lights in the cluster;
b. wherein the clusters of each row have consistent light placement configuration with respect to clusters within the same row; and
c. wherein the clusters of a first row have a different configuration than the clusters of a second row.

12. The dimmable ultraviolet lamp of claim 11, further comprising a third row, wherein:
a. the clusters of the first row include two UVC LED lights, one UVA LED light, and one UVB LED light;
b. the clusters of the second row include two UVA LED lights, one UVB LED light, and one UVC LED light; and
c. the clusters of the third row include two UVB LED lights, one UVA LED light, and one UVC LED light.

13. The dimmable ultraviolet lamp of claim 12, wherein the UV lights are selectably dimmable at least one of: individually, by cluster, or by row.

14. The dimmable ultraviolet lamp of claim 13, further comprising:
a. an occupancy sensor, in communication with the power source, wherein in a negative mode no occupants are sensed by the occupancy sensor, and in a positive mode occupants are sensed by the occupancy sensor, and
b. wherein the control module including instructions for automatically dimming the UV emitters when the occupancy sensor is in a positive mode but not dimming all emitters the same amount.

15. The dimmable ultraviolet lamp of claim 12, further comprising a wireless controller, in wireless communication with the UV lights, and in communication with the power source, wherein the wireless controller has an on-mode and an off-mode.

16. The dimmable ultraviolet lamp of claim 12, further comprising a printed circuit board disposed within the housing, in communication with the power source, and coupled to the UV lights.

17. The dimmable ultraviolet lamp of claim 16, further comprising a plurality of heat sinks disposed behind the printed circuit board opposite the UV lights.

18. The dimmable ultraviolet lamp of claim 17, further comprising a plurality of optics disposed in front of the UV emitters, opposite the heat sinks.

19. An ultraviolet lamp, comprising:
a. a lamp housing;
b. a plurality of rows of ultraviolet (UV) light emitters, coupled to the housing and disposed linearly and longitudinally along a single face of the lamp housing;
i. wherein each row includes an array of clusters of UV light emitters, each cluster including a diverse set of UV light emitters, wherein the light emitters are diverse in the frequency spectrum of light emission between at least two of the emitters in the cluster;
ii. wherein the clusters of each row have consistent light emitter placement configuration with respect to clusters within the same row; and
iii. wherein the clusters of a first row have a different configuration than the clusters of a second row;
c. a third row;
i. wherein the clusters of the first row include two UVC LED lights, one UVA LED light, and one UVB LED light;
ii. wherein the clusters of the second row include two UVA LED lights, one UVB LED light, and one UVC LED light; and
iii. wherein the clusters of the third row include two UVB LED lights, one UVA LED light, and one UVC LED light;
d. a power source coupled to the lamp housing opposite the single face and in functional communication with the UV light emitters; and
e. a wireless controller, in wireless communication with the UV emitters, and in communication with the power source, wherein the wireless controller has an on-mode and an off-mode;
i. wherein the UV emitters are selectably dimmable at least one of: individually, by cluster, or by row.

20. The ultraviolet lamp of claim 19, further comprising:
a. an occupancy sensor, in communication with the power source, wherein in a negative mode no occupants are sensed by the occupancy sensor, and in a positive mode occupants are sensed by the occupancy sensor, and
b. a control module including instructions for automatically dimming the UV emitters when the occupancy sensor is in a positive mode but not dimming all emitters the same amount.
